(19) United States
(12) Patent Application Publication
(10) Pub. No.: US 2024/0277755 A1
(43) Pub. Date: Aug. 22, 2024

(54) Hypertension Preload Treatment and Method

(71) Applicant: Yao-Foli Sekyema, Greensboro, NC (US)

(72) Inventor: Yao-Foli Sekyema, Greensboro, NC (US)

(21) Appl. No.: 18/444,852

(22) Filed: Feb. 19, 2024

Related U.S. Application Data
(60) Provisional application No. 63/446,950, filed on Feb. 20, 2023.

(51) Int. Cl. A61K 31/745; A61B 5/00; A61B 5/021; A61K 9/00

(52) U.S. Cl. CPC A61K 31/745; A61B 5/021; A61B 5/4848; A61K 9/0053

(57) ABSTRACT

A hypertension preload treatment formulation for limiting ingested sodium from entering the bloodstream and method of using the same. The hypertension preload treatment formulation is provided in an oral dosage from. The hypertension preload treatment formulation comprises a therapeutically effective dosage of a sodium-binding agent and an excipient. The sodium-binding agent is a resin comprising a polystyrene cross-linked chain structure formulated to sequestering sodium cations in the resin for later excretion. Methods of binding dietary sodium and treating or preventing hypernatremia in a hypertensive patient are also provided.

### CROSS-REFERENCE TO RELATED APPLICATION

The present application claims priority to, and the benefit of, U.S. Provisional Application No. 63/446,950, which was filed on Feb. 20, 2023, and is incorporated herein by reference in its entirety.

### FIELD OF THE INVENTION

The present invention generally relates to a unique way to treat hypertension. More specifically, the present invention relates to a novel way for treating high blood pressure by inhibiting ingested sodium from entering the bloodstream. Accordingly, the present specification makes specific reference thereto. However, it is to be appreciated that aspects of the present invention are also equally amenable to other like applications, devices and methods of manufacture.

### BACKGROUND

Hypertension is highly prevalent health condition affecting up to 30-45% of adults world-wide. It can lead to stroke, heart attack, heart failure and kidney failure, which are all leading causes of death among affected individuals. Existing treatment methods for hypertension that rely on body sodium load reduction, only target the sodium after it has entered the body, which can be incredibly difficult and expensive.

Hypertension or high blood pressure, is a medical condition in which the blood pressure in the arteries is constantly elevated. High blood pressure by itself usually does not cause obvious symptoms. Long-term high blood pressure, however, is a major risk factor for many chronic conditions, such as, stroke, coronary artery disease, heart failure, atrial fibrillation, peripheral arterial disease, vision loss, chronic kidney disease, and dementia. Hypertension is a major cause of premature death throughout the entire world.

High blood pressure is classified as primary or secondary hypertension. Most cases are primary hypertension, defined as high blood pressure due to nonspecific lifestyle and genetic factors. Lifestyle factors that increase the risk include excess salt in the diet, excess body weight, smoking, and alcohol use. The remaining cases are categorized as secondary high blood pressure, defined as high blood pressure due to an identifiable cause, such as a chronic disease.

Blood pressure is classified the measurements of systolic and diastolic pressures, which are the maximum and minimum pressures, respectively. For most adults, normal blood pressure at rest is within the range of 100-130 millimeters mercury (mmHg) systolic and 60-80 mmHg diastolic. High blood pressure is typically defined as when the resting blood pressure is persistently at or above 130/80 or 140/90 mmHg.

Lifestyle changes and medications can lower blood pressure and decrease the risk of health complications from hypertension. Lifestyle changes include weight loss, physical exercise, decreased salt intake, reducing alcohol intake, and having a healthy diet. If lifestyle changes are not sufficient, then blood pressure medications are employed to attempt to control the pressures.

In most people with established essential hypertension, increased resistance to blood flow (total peripheral resistance) accounts for the high pressure while cardiac output remains normal. Many mechanisms have been proposed to account for the rise in peripheral resistance in hypertension. There is significant evidence that implicates either disturbances in the kidneys' salt and water handling or abnormalities of the sympathetic nervous system. These mechanisms are not mutually exclusive and it is likely that both contribute to some extent in most cases of essential hypertension. Excessive sodium in the diet can lead to excessive intracellular sodium, which contracts vascular smooth muscle, restricting blood flow and increasing blood pressure.

Several classes of medications, collectively referred to as antihypertensive medications, are typically used for treating hypertension. First-line medications include thiazide-diuretics, calcium channel blockers, angiotensin converting enzyme inhibitors, and angiotensin receptor blockers. These medications may be used alone or in combination. Most people require more than one medication to control their hypertension. All of these medication work in some way to attempt to regulate the metabolism of sodium once it is absorbed into the blood stream. There are no current therapeutic approaches to prevent ingested sodium from entering the bloodstream before it is a problem.

Therefore, there exists a long felt need in the art for a medication that restricts sodium from entering the bloodstream. There is also a long felt need for a way to treat high blood pressure by reducing sodium entering the bloodstream directly. Additionally, there is a long felt need in the art for a way to reduces the risk of diseases and conditions associated with hypertension. Finally, there is a long felt need in the art for a hypertension delivery medication in a variety of oral forms.

In this manner, the improved hypertension relief medication of the present invention accomplishes all of the foregoing objectives, thereby providing an easy solution to limit sodium from entering the bloodstream. A primary feature of the present invention is a medication and method that helps treat high blood pressure by reducing sodium entering the bloodstream rather than seeking to target sodium after being absorbed in the body. The present invention is designed to reduces the risk of heart failure, stroke, kidney failure, and other conditions associated with hypertension. Finally, the improved hypertension delivery medication of the present invention is capable of allowing users to take the medication via powder, liquid, capsule, and other means.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a hypertension preload treatment medication that limits the amount of sodium entering the bloodstream. The medication binds dietary sodium in the gut and digestive system of the user via an ion exchange compound to decrease the absorption of sodium into the bloodstream of the body. The medication may be in the form of a powder, liquid suspension, capsule, or pill.

The hypertension preload treatment medication is provided as a sodium removing composition. The sodium removing composition comprises an ion-exchange compound. The ion-exchange compound is a cation-exchange resin. The cation-exchange resin comprises a polystyrene cross-linked chain structure. The polystyrene cross-linked chain structure comprises a plurality of compatible cations. The plurality of compatible cations are each formulated to exchange with a sodium cation allowing the exchanged sodium cations to become sequestered in the cation-exchange resin. The polystyrene cross-linked chain structure further comprises a plurality of divalent bond bridges. The plurality of divalent bond bridges are arranged to bond a plurality of polystyrene chains together to form the polystyrene cross-linked chain structure.

The ion-exchange compound is formulated to bind dietary sodium in a mammalian gut prior to absorption in the bloodstream. Once bonded to the cation-exchange resin, the bonded sodium and cation-exchange resin can no longer be absorbed via the digestive system. Then the bonded sodium and cation-exchange resin is excreted in feces.

The sodium removing composition further comprise an excipient portion. The excipient portion may comprise a preservative. The excipient portion may further comprise a solvent. The excipient portion may further comprise a flavoring. The excipient portion may further comprise a coating. The sodium removing composition is formulated for oral administration and may be formulated as a liquid, a pill, or a capsule.

An additional embodiment of the present invention comprises a method of binding dietary sodium in a human subject. The method comprises administering a sodium removing composition to the human subject. The dosage is provided as an oral dosage. The method continues by determining an effectiveness of the sodium removing composition. The effectiveness is determined by measuring a blood-sodium level in the human subject. The effectiveness may be further measured by further measured by comparing blood pressure readings before and after administration of the sodium removing composition.

The sodium removing composition is a cation-exchange resin. The cation-exchange resin comprises a polystyrene cross-linked chain structure. The polystyrene cross-linked chain structure comprises a plurality of compatible cations. Each of the plurality of compatible cations is formulated to exchange with a sodium cation thereby sequestering the sodium cation in the cation-exchange resin.

An additional embodiment of the present invention comprises a method of treating or preventing hypernatremia in a hypertensive patient. The method comprises administering an effective amount of a sodium-binding agent to the patient. The sodium-binding agent is administered orally. The method continues by determining an effectiveness of treatment. The effectiveness is measured by measured by comparing blood pressure readings before and after treatment. The effectiveness is may also be determined by measuring a blood-sodium level in the patient.

The sodium-binding agent is a cross-linked polystyrene cation exchange resin. The cross-linked polystyrene cation exchange resin comprises a plurality of compatible cations. Each of the plurality of compatible cations is formulated to exchange with a sodium cation thereby sequestering the sodium cation in the sodium-binding agent. The cross-linked polystyrene cation exchange resin is a plurality of polystyrene chains is bonded together via a plurality of divalent bond bridges to form the polystyrene cross-linked chain structure.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic representation of one embodiment of an ion exchange mechanism of a sodium removing composition of the present invention in accordance with the disclosed architecture.

FIG. 2 illustrates a schematic structure a cation-exchange resin of the sodium removing composition of the present invention in accordance with the disclosed architecture.

FIG. 3 illustrates a schematic representation of a plurality of positively charged cations exchanging with a plurality of sodium cations passing through the cation-exchange resin of the sodium removing composition of the present invention in accordance with the disclosed architecture.

FIG. 4 illustrates a schematic representation an exchanging charges using an ion-exchange resin column of the sodium removing composition of the present invention in accordance with the disclosed architecture.

FIG. 5 illustrates a schematic structure an arrangement of the cation-exchange resin of the sodium removing composition of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is a treatment that limits the amount of sodium entering the bloodstream. High sodium content in blood is the predominant cause of hypertension in most people. Current medications target sodium after it has been absorbed into the bloodstream. The medication compound of the present invention binds dietary sodium in the gut and digestive system of the user via an ion exchange compound to decrease the absorption of sodium into the bloodstream of the body. The treatment may be in the form of a powder, liquid suspension, capsule, or pill.

The treatment is a hypertension preload treatment formulation for limiting ingested sodium from entering the bloodstream. The hypertension preload treatment formulation is provided in an oral dosage from. The hypertension preload treatment formulation comprises a therapeutically effective dosage of a sodium-binding agent and an excipient. The sodium-binding agent is a resin comprising a polystyrene cross-linked chain structure formulated to sequestering sodium cations in the resin for later excretion. The treatment is also provided as a method of binding dietary sodium and treating or preventing hypernatremia in a hypertensive patient.

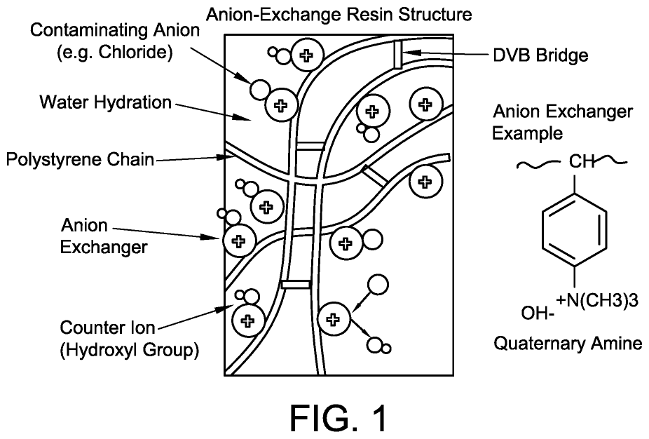

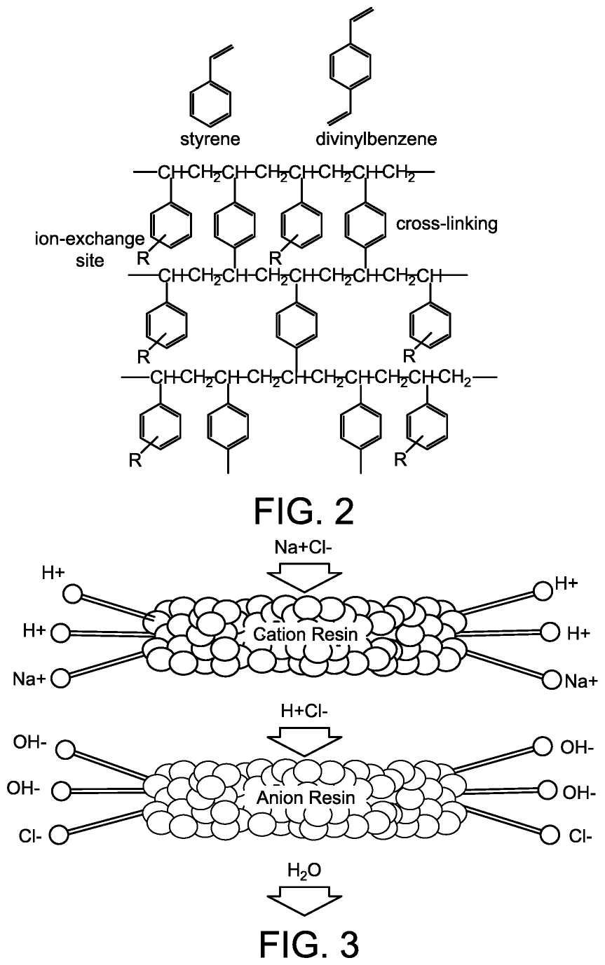

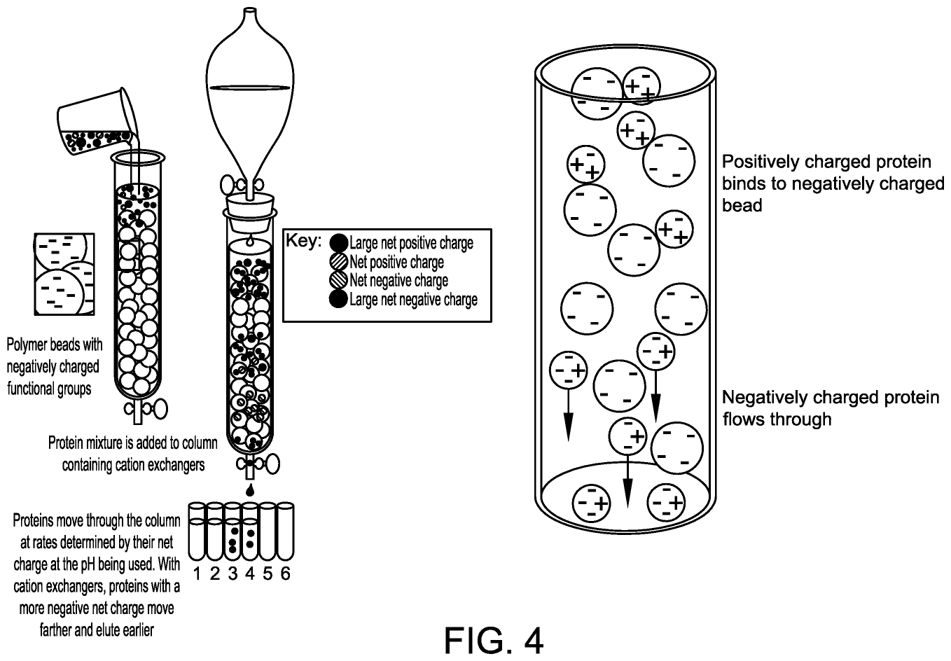

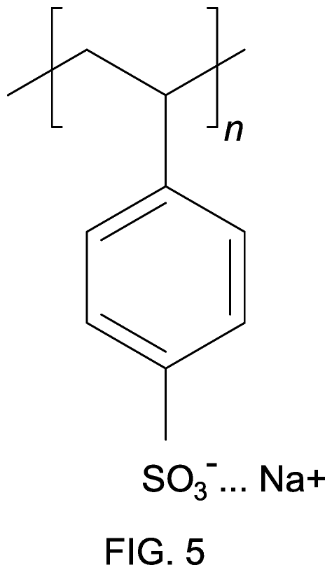

Referring initially to the drawings, FIGS. 1-5 illustrates an ion exchange mechanism of a sodium removing composition of the present invention. The present invention, in one exemplary embodiment, comprises a hypertension preload treatment medication. The hypertension preload treatment medication is formulated to limit the amount of ingested sodium that can enter the bloodstream. The medication is designed to bind with dietary sodium in the gut and digestive system of the user. The medication provides an ion exchange compound designed sequester the sodium and to decrease the absorption of the sodium into the bloodstream of the body. The medication may be in the form of a powder, liquid suspension, capsule, or pill for oral consumption.

The hypertension preload treatment medication is provided as a sodium removing composition. The sodium removing composition comprises an ion-exchange compound. The ion-exchange compound is a cation-exchange resin. An ion-exchange resin is a resin often configured as an insoluble matrix medium for ion exchange.

The cation-exchange resin comprises a polystyrene cross-linked chain structure. The polystyrene cross-linked chain structure comprises a plurality of compatible cations. The plurality of compatible cations are each formulated to exchange with a sodium cation allowing the exchanged sodium cations to become sequestered in the cation-exchange resin. The polystyrene cross-linked chain structure further comprises a plurality of divalent bond bridges. The plurality of divalent bond bridges are arranged to bond a plurality of polystyrene chains together to form the polystyrene cross-linked chain structure. The polystyrene is crosslinked, which keeps the resin from dissolving. The plurality of divalent bond bridges are the cross-link bonds that hold the polystyrene chains together.

The ion-exchange compound is formulated to bind dietary sodium in a mammalian gut prior to absorption in the bloodstream. Once bonded to the cation-exchange resin, the bonded sodium and cation-exchange resin can no longer be absorbed via the digestive system. Then the bonded sodium and cation-exchange resin is excreted in feces. Less sodium entering the bloodstream will limit the amount of water drawn from the intracellular spaces into the bloodstream. This will make the space less tanked or engorged, thus keeping blood pressure lower.

The sodium removing composition further comprise an excipient portion. The excipient portion may comprise a preservative. The preservative may be Methyl Paraben, Ethyl Paraben, PropylParaben, Phenol, Benzoic acid, or any similar preservative used in medications. The excipient portion may further comprise a solvent. The solvent may be ethyl alcohol, liquid polyethylene glycol, and propylene glycol, or any similar solvent used in medications. The excipient portion may further comprise a flavoring. The flavoring may be aspartame, mannitol, sorbitol, sucrose, maltose, sucralose, or any similar flavoring used in medications. The excipient portion may further comprise a coating. The flavoring may be a gelatine, plasticizer, a sugar coating, a film coating, an enteric coating, or any similar coating used in medications. The sodium removing composition is formulated for oral administration and may be formulated as a liquid, a pill, or a capsule.

An additional embodiment of the present invention comprises a method of binding dietary sodium in a human subject. The method comprises administering a sodium removing composition to the human subject. The dosage is provided as an oral dosage. The method continues by determining an effectiveness of the sodium removing composition. The effectiveness is determined by measuring a blood-sodium level in the human subject. The effectiveness may be further measured by further measured by comparing blood pressure readings before and after administration of the sodium removing composition. The sodium removing composition may be administered daily or as needed based on effectiveness.

The sodium removing composition is a cation-exchange resin. The cation-exchange resin comprises a polystyrene cross-linked chain structure. The polystyrene cross-linked chain structure comprises a plurality of compatible cations. Each of the plurality of compatible cations is formulated to exchange with a sodium cation thereby sequestering the sodium cation in the cation-exchange resin. The polystyrene cross-linked chain structure further comprises a plurality of divalent bond bridges. The plurality of divalent bond bridges are arranged to bond a plurality of polystyrene chains together to form the polystyrene cross-linked chain structure.

The cation-exchange resin is formulated to bind dietary sodium in a mammalian gut prior to absorption in the bloodstream. Once bonded to the cation-exchange resin, the bonded sodium and cation-exchange resin can no longer be absorbed via the digestive system. Then the bonded sodium and cation-exchange resin is excreted in feces.

The sodium removing composition further comprise an excipient portion. The excipient portion may comprise a preservative. The excipient portion may further comprise a solvent. The excipient portion may further comprise a flavoring. The excipient portion may further comprise a coating. The sodium removing composition is formulated for oral administration and may be formulated as a liquid, a pill, or a capsule.

An additional embodiment of the present invention comprises a method of treating or preventing hypernatremia in a hypertensive patient. The method comprises administering an effective amount of a sodium-binding agent to the patient. The sodium-binding agent is administered orally. The method continues by determining an effectiveness of treatment. The effectiveness is measured by measured by comparing blood pressure readings before and after treatment. The effectiveness is may also be determined by measuring a blood-sodium level in the patient. The sodium-binding agent may be administered daily or as needed based on effectiveness.

The sodium-binding agent is a cross-linked polystyrene cation exchange resin. The cross-linked polystyrene cation exchange resin comprises a plurality of compatible cations. Each of the plurality of compatible cations is formulated to exchange with a sodium cation thereby sequestering the sodium cation in the sodium-binding agent. The cross-linked polystyrene cation exchange resin is a plurality of polystyrene chains is bonded together via a plurality of divalent bond bridges to form the polystyrene cross-linked chain structure. The plurality of divalent bond bridges are arranged to bond a plurality of polystyrene chains together to form the polystyrene cross-linked chain structure.

The sodium-binding agent is formulated to bind dietary sodium in a mammalian gut prior to absorption in the bloodstream. Once bonded to the cross-linked polystyrene cation exchange resin, the bonded sodium and cross-linked polystyrene cation exchange resin can no longer be absorbed via the digestive system. Then the bonded sodium and cross-linked polystyrene cation exchange resin is excreted in feces.

The sodium removing composition further comprise an excipient portion. The excipient portion may comprise a preservative. The excipient portion may further comprise a solvent. The excipient portion may further comprise a flavoring. The excipient portion may further comprise a coating. The sodium removing composition is formulated for oral administration and may be formulated as a liquid, a pill, or a capsule.

It is contemplated that the sodium removing composition constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sodium removing composition comprising:
an ion-exchange compound; and
an excipient portion; and
wherein the ion-exchange compound is a cation-exchange resin comprising a polystyrene cross-linked chain structure comprising a plurality of compatible cations each formulated to exchange with a sodium cation thereby sequestering the sodium cation in the cation-exchange resin.

2. The sodium removing composition of claim 1, wherein the sequestering sodium cation in the cation-exchange resin is excreted in feces.

3. The sodium removing composition of claim 1, wherein the excipient portion comprises a preservative.

4. The sodium removing composition of claim 1, wherein the excipient portion comprises a solvent.

5. The sodium removing composition of claim 1, wherein the excipient portion comprises a flavoring.

6. The sodium removing composition of claim 1 further comprising a coating.

7. The sodium removing composition of claim 6, wherein the coating is a gelatin or a plasticizer.

8. The sodium removing composition of claim 1, wherein the ion-exchange compound is formulated to bind with dietary sodium in a mammalian digestive system prior to absorption into the bloodstream.

9. The sodium removing composition of claim 1, wherein the polystyrene cross-linked chain structure further comprises a plurality of divalent bond bridges arranged to bond a plurality of polystyrene chains together to form the polystyrene cross-linked chain structure.

10. The sodium removing composition of claim 1, wherein the sodium removing composition is formulated as a liquid.

11. The sodium removing composition of claim 1, wherein the sodium removing composition is formulated as pill.

12. The sodium removing composition of claim 1, wherein the sodium removing composition is formulated as capsule.

13. A method of binding dietary sodium in a human subject, the method comprising:
administering a sodium removing composition in an oral dosage to the human subject;
wherein the sodium removing composition is a cation-exchange resin comprising a polystyrene cross-linked chain structure comprising a plurality of compatible cations each formulated to exchange with a sodium cation thereby sequestering the sodium cation in the cation-exchange resin;
determining an effectiveness of the sodium removing composition by measuring a blood-sodium level.

14. The method of claim 13, wherein the effectiveness of treatment is further measured by comparing blood pressure readings before and after administration.

15. The method of claim 13, wherein the sequestering sodium cations are excreted in feces.

16. The method of claim 13, wherein the sodium removing composition is formulated to bind with dietary sodium in a human digestive system prior to absorption into the bloodstream.

17. A method of treating hypernatremia in a hypertensive patient, the method comprising;
administering an effective amount of a sodium-binding agent to the patient;
wherein the sodium-binding agent is a cross-linked polystyrene cation exchange resin comprising a plurality of compatible cations each formulated to exchange with a sodium cation thereby sequestering the sodium cation in the sodium-binding agent;
measuring an effectiveness of treatment by comparing blood pressure readings before and after treatment.

18. The method of claim 17, wherein the sodium-binding agent is administered daily.

19. The method of claim 17, wherein the cross-linked polystyrene cation exchange resin is a plurality of polystyrene chains is bonded together via a plurality of divalent bond bridges to form the polystyrene cross-linked chain structure.

20. The method of claim 17, wherein the sodium-binding agent is administered orally.
